(19) United States
(12) Patent Application Publication
(10) Pub. No.: US 2018/0169010 A1
(43) Pub. Date: Jun. 21, 2018

(54) LONG ACTING LIRAGLUTIDE COMPOSITIONS

(71) Applicant: Sun Pharma Advanced Research Company Limited, Mumbai (IN)

(72) Inventors: Ajay Jaysingh Khopade, Baroda (IN); Arindam Halder, Baroda (IN); Vivek Patel, Baroda (IN)

(73) Assignee: Sun Pharma Advanced Research Company Limited, Mumbai (IN)

(21) Appl. No.: 15/736,396

(22) PCT Filed: Jun. 16, 2016
(86) PCT No.: PCT/IN2016/050185

§ 371 Date: Dec. 14, 2017

(30) Foreign Application Priority Data
Jun. 16, 2015 (IN) 1567/MUM/2015

(51) Int. Cl. A61K 9/06; A61K 38/26; A61K 9/00; A61K 47/10; A61K 47/34; A61K 47/14

(52) U.S. Cl. CPC A61K 9/06; A61K 38/26; A61K 47/14; A61K 47/10; A61K 47/34; A61K 9/0019

(57) ABSTRACT

The present invention relates to a long acting composition comprising, therapeutically effective amount of liraglutide, a block or a graft copolymer comprising hydrophilic and hydrophobic moieties or mixtures thereof, at least one amphipath, and an aqueous vehicle, wherein the composition is in the form of a gel which is rendered injectable when forced through a needle by means of a plunger. The composition provides effective blood glucose control for about 6 days to about 14 days after single administration to a subject in need thereof, in particularly for about a week.

### FIELD OF THE INVENTION

The invention relates to long acting compositions comprising liraglutide, methods of making them and use of such composition in the treatment of metabolic disease

### BACKGROUND OF THE INVENTION

Diabetic mellitus is a disease of metabolic dysregulation, most notably abnormal glucose metabolism, accompanied by characteristic long term complications. It's a chronic disease requiring long term medications. Different parenteral anti-diabetic medications are available in market including human insulin and different GLP-1 agonists.

The natural GLP-1 is a gut hormone with therapeutic potential in the treatment of Type 1 and Type 2 diabetes and the treatment of obesity. The natural GLP-1 has a short half-life of only few minutes in the body as it is rapidly degraded by dipeptidyl peptidase-4 enzyme. Similarly, insulin also has a short half-life and is to be administered once or twice daily to diabetic patients.

Many GLP-1 agonists and insulin analogues were developed by modifications to natural GLP-1 and insulin resp. to overcome the problem of its short half-life. One of the approaches used was substitution of one or more amino acid and attachment of a lipophilic substituent to these peptides. These lipophilic substituted GLP-1 agonists and insulin or insulin analogues showed protracted action when injected.

U.S. Pat. No. 5,750,497 discloses lipophilic substituted insulin including, insulin detemir, wherein human insulin is acylated to myristic acid at B29 lysine. It is administered as once or twice daily subcutaneous injection.

U.S. Pat. No. 7,615,532 discloses lipophilic substituted insulin analogues. On particular example is insulin degludec, wherein desB30 human insulin is conjugated to hexadecanedioic acid via gamma-L-glutamyl spacer at B29. It is administered as once daily subcutaneous injection.

U.S. Pat. No. 6,268,343 disclosed such fatty acid acylated GLP-1 agonists. One particular example includes liraglutide. Liraglutide is a once daily human GLP-1 analog (97% homology). Liraglutide is (Arg34, Lys²⁶ (N^{ϵ}-(γ-GLu(N-hexadecanoyl)))-GLP-1(7-37). In US, Liraglutide is approved as a once daily subcutaneous injection to improve glycemic control in adults with type 2 diabetes mellitus. It is also approved in US for weight management in adult patients.

WO06/097537 discloses acylated GLP-1 analogs including semaglutide, a monoacylated GLP-1 agonist (94% homology) for once weekly administration. Semaglutide has amino acid substitutions at position 8 (alanine to alpha-aminoisobutyric acid, a synthetic amino acid) and position 34 (lysine to arginine), and acylation of the peptide backbone with a spacer and C-18 fatty di-acid chain to lysine at position 26 of the GLP-1. Semaglutide is currently in phase III clinical development for the treatment of Type 2 diabetes mellitus.

Exenatide, a 39 amino acid peptide, is a modified GLP-1 agonist which is available as a twice daily injection for the treatment of Type 2 diabetes mellitus. An extended release formulation of exenatide for once weekly administration is approved in US in 2012. This once-weekly formulation consists of exenatide encapsulated in microspheres. US20140220134 disclosed once monthly formulation of exenatide using extended release microspheres suspension comprising poly (lactide-co-glycolide) polymer in a medium chain triglycerides.

However, there is s still a need to lower the frequency of injections for the patients and to provide a long acting composition for once weekly administration, preferably for once monthly administration.

### SUMMARY OF THE INVENTION

The present inventors have found long acting compositions of lipophilic derivatives of GLP-1 agonist like, liraglutide. Particularly, the present invention provides a long acting composition comprising
a) therapeutically effective amount of liraglutide
b) a block or a graft copolymer comprising hydrophilic and hydrophobic moieties or mixtures thereof,
c) at least one amphipath, and
d) an aqueous vehicle.
   wherein the composition is in the form of a gel which is rendered injectable when forced through a needle by means of a plunger. More particularly, the composition of the invention provides a long acting composition of liraglutide for once weekly administration.

### BRIEF DESCRIPTION OF THE FIGURES

FIG. 1(a), (b), (c), (d), (e), depicts interaction of Liraglutide and Exenatide with Poloxamer. FIG. 1(a) depicts solution formation of Liraglutide with Tromethamine in Water for injection (WFI) in a glass vial. 1(b) depicts solution formation of Poloxamer 188 in WFI, 1(c) depicts solution formation of Liraglutide with Tromethamine and Polysorbate 80 in WFI. 1(d) depicts gel formation of Liraglutide with Tromethamine and Poloxamer 188 in WFI. 1(e) depicts that Exenatide with Poloxamer 188 in WFI remains a solution and does not gel.

FIG. 2(a), (b), (c), (d), (e), depicts interaction of Liraglutide and Exenatide with Soluplus®, 2(a) depicts solution formation of Liraglutide with Tromethamine in WFI in a glass vial, 2(b) depicts solution formation of Soluplus® in WFI, 2(c) depicts solution formation of Soluplus® with Tromethamine in WFI, 2(d) depicts gel formation of Liraglutide with Tromethamine and Soluplus® in WFI. 2(e) depicts Exenatide with Soluplus® in WFI remains a solution and does not gel.

FIG. 3. depicts Cryo-TEM image of composition of Example 4, when analyzed as per Example 6.

FIG. 4. depicts Cryo-TEM image of composition of Example 5, when analyzed as per Example 6.

FIG. 5 provides the preclinical efficacy data in db/db mice comparing Example 1 and Example 4.

FIG. 6 to FIG. 9 provide preclinical efficacy data in db/db mice with weekly administration of Example 4 and Example 7 for 28 days, as compared to daily administration of Victoza®, as determined in Example 11.

### DETAILED DESCRIPTION OF THE INVENTION

The present invention provides long acting compositions of liraglutide, method of treatment using said compositions as well as method of making the same. The composition of the invention can be used in the treatment of metabolic diseases such as diabetes and obesity.

The present inventors have advantageously discovered a long acting gel composition of liraglutide. The composition offers advantage over Victoza® in that it needs to be administered at a lower frequency, thus providing convenience to patients and increasing patient compliance, further providing an effective blood glucose control over a longer period of time. The composition of the invention provides a long acting composition of liraglutide for once weekly administration. Further, the present inventors have found that in spite of a high consistency the gel composition of the present invention acquires sufficient flow characteristics when injected through a needle. Further, the composition regains its consistency at the site of injection, thereby providing a long duration of control on the blood glucose levels.

In one aspect, the present invention provides a long acting composition, comprising
a) therapeutically effective amount of liraglutide,
b) a block or a graft copolymer comprising hydrophilic and hydrophobic moieties or mixtures thereof,
c) at least one amphipath, and
d) an aqueous vehicle.
   wherein the composition is in the form of a gel which is rendered injectable when forced through a needle by means of a plunger.

The term “long acting” as used herein, refers to the duration of action of composition of the liraglutide as disclosed and claimed herein. More specifically, it refers to the period of time after administration of a dose of liraglutide composition of the present invention for which blood glucose levels are controlled. The composition of the present invention provides effective blood glucose control for about 6 days to about 14 days after single administration to a subject in need thereof. In a more preferred embodiment, the composition of the present invention provides effective blood glucose control for about a week after single administration such that the composition may be administered as once a week injection.

Liraglutide may be present in the composition in the form of base or in the form of its salts or mixtures thereof. Representative examples of salts includes salts with suitable inorganic acids such as hydrochloric, hydrobromic, and the like. Representative examples of salts also includes salts with organic acids such as formic acid, acetic acid, propionic acid, lactic acid, tartaric acid, ascorbic acid and the like. Representative examples of salts also includes salt with base such as .triethanolamine, diethylamine, meglumine, lysine, arginine, alanine, leucine, diethylethanolamine, olamine, triethylamine, tromethamine, choline, trimethylamine, taurine, benzamine, methylamine, diemthylamine, trimethylamine, methylethanolamine, propylamine, isopropylamine, and like. In a preferred embodiment, liraglutide may be used in the form of liraglutide acetate. Further the term “liraglutide” also include a mixture of liraglutide base with small amounts of acetic acid for eg. acetic acid may be present in less than 3% of weignt of liraglutide and the present invention includes such form of liraglutide. Such forms of liraglutide are commercially available.

The concentration of liraglutide in the composition of the present invention may be in the range from about 3% to 20% of total weight of the composition. Preferably, the composition comprises liraglutide in a concentration in the range from about 3% to 15% of total weight of the composition. More preferably liraglutide is present at a concentration in the range from about 7% to 10%. It is to be noted that liraglutide has an auxiliary function in that, it being by itself a polymer of 32 amino acids which is derivatized with a lipophilic chain, it contributes to the viscous nature of the composition of the present invention.

The block copolymer that may be used according to the present invention may be selected from polyoxyethylene-polyoxypropylene block copolymer, poly(vinyl alcohol) and poly(ethylene glycol) copolymer, poly(methyl methacrylate) and poly(ethylene glycol) copolymer, poly(methylmethacrylate) and poly(methacrylic acid) copolymer, poly(lactic acid) and poly(ethylene glycol) copolymer, Poly(ethyleneoxide)-poly(butadiene) copolymer, Poly(ethyleneoxide)-Poly(ethylene ethylene) copolymer, 2-methacryloyloxyethyl phosphorylcholine (MPC™) and n-butyl methacrylate (BMA, poly(2-(dimethylamino)ethyl methacrylate) (PDMAEMA) and poly(methyl methacrylate) (PMMA), Dextran-block-poly(E-caprolactone) (DEX-b-PCL) (amphiphilic diblock copolymer), PolyVivo mPEG-PLGA diblock copolymers, PolyVivo mPEG-PCL diblock copolymer, PLGA-block-PEG-block-PLGA) -(poly(lactic acid-co-glycolic acid)-block-poly(ethylene glycol)-block-poly(lactic acid-co-glycolic acid) triblock copolymers, mPEG-PLLA (Methoxy poly(ethylene glycol)-b-poly(L-lactide)) diblock copolymers, mPEG-PDLLA (Methoxy poly(ethylene glycol)-b-poly(D,L-lactide)) diblock copolymers, mPEG-PS (Methoxy poly(ethylene glycol)-b-poly(styrene)) diblock copolymers, mPEG-P(5BZTMC) (Methoxy poly(ethylene glycol)-poly(5-benzyloxy-trimethylene carbonate)) copolymers, mPEG-PTMC (Methoxy poly(ethylene glycol)-poly(trimethylene carbonate)) copolymers, PCL-PEG-PCL (Poly(caprolactone)-b-poly(ethylene glycol)-b-poly(caprolactone)) copolymers, PLA-PCL-PEG-PCL-PLA (Poly(D,L) lactide-b-Poly(caprolactone)-b-Poly(ethylene glycol)-b-Polycaprolactone-b-Poly(D,L)lacide) copolymers, PLA-PEG-PLA (Poly(D,L-lactide)-b-poly(ethylene glycol)-b-poly(D,L-lactide)) copolymers, PDLLA-PEG-PDLLA (Poly(D,L-lactide)-b-poly(ethylene glycol)-b-poly(D,L-lactide)) copolymers, PEG-PDLLA-Decyl(Poly(ethylene glycol)-b-poly(D,L-lactic acid)-decyl) copolymers, Kollidon VA 64 poly(vinylpyrrolidone-vinylacetate copolymer), Poly(N-vinyl-2-pyrrolidone)-poly(D,L-lactide), Poly(2-ethyl-2-oxazline)-block-(poly(epsilon-caprolactone) copolymer, Poly(ethylene oxide)-poly(butylene oxide) di and triblock copolymers, Polystyrene-poly(ethylene oxide) di and triblock copolymers, Poly(2-methyl-2-oxazoline)-b-poly(2-alkyl-2-oxazoline), Poly(methyl methacrylate)-poly(ethylene oxide) di block copolymer, Poly(N-isopropyl acrylamide)-Poly(y-benzyl-L-glutamate), Poly(ethylene oxide)-Poly(methylidene malonate), Poly(ethylene oxide)-poly(acrylic acid), Poly(ethylene oxide)-poly(methacrylic acid), Poly(ethylene oxide)-poly(vinyl benzoate), Poly(ethylene oxide)-Poly(N-isopropylacrylamide), Poly(ethylene oxide)-Poly(2-vinyl pyridine), Poly(vinyl benzyl alcohol)-Poly(oligo(ethylene glycol)methacrylate), Polystyrene-poly(acrylic acid), Polystyrene-poly(methacrylic acid) and Poly(2-ethoxyethyl vinyl ether)-Poly(2-methoxy ethyl vinyl ether) or mixtures thereof.

In a preferred embodiment, the block copolymer that may be used in the present composition may be a polyoxyethylene-polyoxypropylene block copolymer. Such copolymers are available commercially as Poloxamers of different grades, for eg. poloxamer 105, poloxamer 108, poloxamer 122, poloxamer 123, poloxamer 181, poloxamer 188, poloxamer 212, poloxamer 217, poloxamer 235, poloxamer 237, poloxamer 282, poloxamer 331, poloxamer 338, poloxamer 401, poloxamer 403 and poloxamer 407 or mixtures thereof. In a preferred embodiment, the block copolymer may be poloxamer 188.

The graft copolymer may be selected from polyvinyl caprolactam-polyvinyl acetate-polyethylene glycol graft copolymer, commercially available as Soluplus®, PUREBRIGHT mb-37-50T and PUREBRIGHT mb-37-100T (2-methacryloyloxyethyl phosphorylcholine (MPCTM) and n-butyl methacrylate (BMA)) copolymers, Kollicoat (PVA-PEG graft copolymer), poly(styrene-co-methyl methacrylate-co-maleic anhydride) and poly(ethylene oxide) monomethyl ether graft copolymer, poly(vinyl alcohol)-poly(ethylene glycol) graft copolymer and poly(N-isopropylacrylamide)-b-[poly(ethyl acrylate)-g-poly (2-vinylpyridine)] or mixtures thereof. In a preferred embodiment, the graft copolymer may be a polyvinyl Caprolactam-polyvinyl acetate and polyethylene glycol-based graft copolymer.

The concentration of the block copolymer or graft copolymer used is sufficient to impart a viscous gel like consistency preferably a semi-solid consistency to the compositions of the present invention. The present inventors have found that inspite of the high consistency, the gel composition of the present invention acquires sufficient flow characteristics when injected through a needle. At the site of the injection, the composition regains its consistency and provides a long duration of control on the blood glucose levels. The block or graft copolymer is present at a concentration from 1% to 5% by weight of the composition. More preferably, polyoxyethylene-polyoxypropylene block copolymer is present at a concentration from 1.5% to 3% by weight of the composition.

The term “amphipath” as used herein refers to compounds which contain both a hydrophilic and a hydrophobic (lipophilic) group Amphipaths suitable for use in the composition include but not limited to mono and diglycerides, polyglycerized fatty acids, polyethoxylated fatty acids, PEG-fatty acid mono and di-esters and mixtures thereof, PEG glycerol fatty acid esters, Alcohol-oil transesterification products, propylene glycol fatty acid esters, mixtures of propylene glycol esters and glycerol esters, PEG sorbitan fatty acid esters, PEG alkyl ethers, PEG alkyl phenols, and sorbitan fatty acid esters

Examples of mono di and triglycerides for use as amphipath in the composition of present invention are glycerol monoleate, glycerol monolaurate, glycerol monopalmitate, glycerol monostearate, glycerol acetate, glycerol laurate, glycerol caprylate, glycerol caprate, glyceryl monostearate, glycerol dioleate, caprylic acid mono, diglycerides, dicaprin, dimyristin, dipalmitin, glyceryl dilaurate, glycerol trioleate, glycerol tristearate and glycerol esters of fatty acids.

Examples of polyglycerized fatty acids for use as amphipath in the composition of present invention are polyglyceryl-2, 4, 10, stearate, polyglyceryl-2, 3, 4, 6, 10 oleate, polyglyceryl-2 isostearate, polyglyceryl-10 laurate, polyglyceryl-6 ricinoleate, polyglyceryl-10 linoleate, polyglyceryl-2, 3 dioleate, polyglyceryl-3 distearate and polyglyceryl-10 trioleate.

Examples of polyethoxylated fatty acids for use as amphipath in the composition of present invention are PEG 1-10 Stearate, PEG 2oleate, PEG 41aurate, PEG 4-100 monooleate, PEG 4-monostearate,

Examples of PEG-fatty acid di-esters and mixtures with mono-esters for use as amphipath in the composition of present invention are diesters of lauric acid, oleic acid, stearic acid, palmitic acid with different grades of PEG such as PEG-4 dilaurate, PEG-4-dioleate, PEG-4 distearate, PEG-10 dipalmitate, PEG-6 dilaurate, PEG-6 dioleate, PEG-6 distearate, PEG-8 dilaurate, PEG-8 dioleate, PEG-8 distearate, PEG-12 dilaurate, PEG-12 dioleate, PEG-12 distearate, PEG-32 dilaurate, PEG-32 dioleate, PEG-32 distearate.

Examples of PEG glycerol fatty acid esters for use as amphipath in the composition of present invention are esters of lauric acid, oleic acid, stearic acid with different grades of PEG such as PEG-15 glyceryl laurate, PEG-20 glyceryl laurate, PEG-20 glyceryl stearate, PEG-20 glyceryl oleate and PEG-15 glyceryl oleate.

Examples of Alcohol-oil transesterification products for use as amphipath in the composition of present invention are PEG-5-10 castor oil, PEG-5, 7, 10 hydrogenated castor oil, PEG-6 peanut oil, PEG-6 kernel oil, PEG-6 corn oil, PEG-20 corn glycerides, PEG-8 and 6 caprylic/capric glycerides, Pentaerythrityl tetraisostearate Pentaerythrityl distearate, Pentaerythrityl tetraoleate, Pentaerythrityl tetrastearate Pentaerythrityl tetracaprylate/tetracaprate.

Examples of propylene glycol fatty acid esters for use as amphipath in the composition of present invention are propylene glycol monocaprylate, propylene glycol monolaurate, propylene glycol oleate, propylene glycol myristate, propylene glycol ricinoleate, propylene glycol dicaprylate/dicaprate, propylene glycol dioctanoate, propylene glycol dilaurate, propylene glycol distearate and propylene glycol dicaprylate.

Examples of mixtures of propylene glycol esters and glycerol esters for use as amphipath in the composition of present invention are esters of oleic and stearic acid.

Examples of PEG sorbitan fatty acid esters for use as amphipath in the composition of present invention are PEG-4, 6, 10 sorbitan monolaurate, PEG-10 sorbitan monopalmitate, PEG-4, 6, 8, 10 sorbitan monostearate, PEG-5, 6, 10 sorbitan monooleate, PEG-6 sorbitan tetraoleate, PEG-6 sorbitan tetrastearate, PEG sorbitan hexaoleate, PEG sorbitan hexastearate.

Examples of PEG alkyl ethers for use as amphipath in the composition of present invention are PEG oleyl ethers, PEG lauryl ethers, PEG cetyl ethers and PEG stearyl ethers.

Examples of PEG alkyl phenols for use as amphipath in the composition of present invention are PEG-10 nonyl phenol and PEG-15 octylphenol ether.

Examples of sorbitan fatty acid esters for use as amphipath in the composition of present invention are sorbitan monopalmitate, sorbitan monooleate, sorbitan monostearate, sorbitan monolaurate, sorbitan trioleate, sorbitan tristearate, sorbitan sesquistearate and sorbitan sesquioleate.

Amphipaths suitable for use in the composition may also include a phospholipid. Phospholipids used in present invention are obtained from plant source, animal sources or synthetic source. Natural phospholipids can be obtained from vegetable sources like, e.g., soybeans, rape (canola) seed, wheat germ, animal material, like egg yolk, milk etc. Examples of natural phospholipids are soya lecithin, egg lecithin, enzyme-modified natural phospholipids such as monoacyl-phosphatidylcholine (lyso PC), soy PE, soy PG, egg PG, and saturated analogs. Examples of synthetic phospholipid are PEG-ylated phospholipids and the cationic phospholipid 1,2-diacyl-P-O-Ethylphosphatidylcholine or mixtures thereof. Examples of semisynthetic phospholipids include Dipalmitoylphosphatidylcholine (DPPC), 1-palmitoyl-2-oleoyl phosphotidylcholine (POPC), dioleoyl phosphotidylcholine (DOPC), dilinoleoyl phosphotidylcholine (DLiPC), lysophosphatidylcholine (LPC), 1-palmityol-LPC (PaLAC), 1-oleoyl-LPC (OiLPC), Phosphotidylethanolomine(PE), plasmenyl ethanolamine (PlaE), glycerol acetal of plasmenyl ethanolamine (GAPlaE), didodecyl phosphatidyl ethanolamine (DDPE), dielaidoyl phosphatidyl ethanolamine (DEPE), dioleoyl phosphatidyl ethanolamine (DOPE), dilinoleoyl phosphatidyl ethanolamine (DLiPE), dioleoyl phosphatidyl-N-monomethyl ethanolamine (DOPE-Me), dophosphotidylglycerol(DPG), Phosphotidylglycerol (PG), Phosphotidylserine (PS), Phosphotidylinositol (PI), Preferred phospholipid includes phosphotidyl choline. Preferably phosphotidyl choline is soy phosphotidyl choline (SPC) or mixtures thereof.

Amphipaths suitable for use in the composition may include nonionic and zwitterionic surfactants, monoglyceride and sphingolipids and phospholipids as described in Fontell et al., Colloidal & Polymer science, 268: 264-285 (1990)

The composition of the invention may be subcutaneous administered and preferably comprises an amphipath wherein the amphipath is a mixture of glyceryl monooleate, glyceryl dioleate, glyceryl trioleate and phosphotidylcholine. More preferably, the pharmacopoeial grade, commercially available amphipath available by the trade names IMWITOR® is used. IMWITOR® 948 is manufactured by esterification of plant derived glycerol with vegetable sourced fatty acids, mainly oleic acid, which contains 40% of nominal content of monoglyceride in ratio of monoglyceride (32.0-52.0%), diglyceride (30.0-50.0%) and triglyceride (5.0-20.0%).

The mixture of mono, di and triglyceride are available in different ratios as per the below nominal content of monoglyceride and available as different grades of IMWITOR®

| | IMWITOR ® | | |
|---|---|---|---|
| | Nominal content of monoglyceride (%) | | |
| | 40 | 60 | 90 |
| Monoglyceride | 32.0-52.0 | 55.0-65.0 | 90.0-101.0 |
| Diglyceride | 30.0-50.0 | 15.0-35.0 | <10.0 |
| Triglyceride | 5.0-20.0 | 2.0-10.0 | <2.0 |

Generally, the weight ratio between a phospholipid and a mixture of a mono, di and triglycerides thereof in the present composition is 50:50. In another aspect, the weight ratio between a phospholipid and a diglyceride like glyceryl dioleate in the present composition is in the range from 20:60 to 30:70.

The amphipath in the present invention is present at a concentration from 50% to 80% by weight of the composition, preferably, 60% to 70% by weight of the composition.

The composition of the present invention comprises an aqueous vehicle. The aqueous vehicle includes water for dissolving water soluble or water miscible components, and at least one water miscible solvent for dissolving amphipaths, particularly amphipaths that are not water soluble. The aqueous vehicle is a mixture of water and a water miscible solvent. An aqueous vehicle suitable for use in the composition include but not limited to water, alcohols, ethers, esters and ketones or mixtures thereof. Alcohols may include class of vehicles and include monols, diols and polyols, for eg. ethanol, glycerol or propylene glycol. Suitable ethers may include diethyl ether, glycofurol, diethylene glycol and polyethylene glycol. Preferably, the aqueous vehicle is selected from water, ethanol, propylene glycol, glycofurol and mixtures thereof. Unlike conventional liquid and semi-solid compositions, the composition of the present has a lower concentration of the liquid vehicle than the total concentration of other components of the invention. The aqueous vehicle is present at a concentration from 20% to 40% by weight of the composition, preferably, at a concentration from 25% to 35% by weight of the composition. The composition may contain water in the concentration from 10% to 25% by weight of the composition, preferably, from 14% to 21% by weight of the composition. Such pharmaceutical compositions of liraglutide with low concentration of aqueous vehicle or water useful as long acting compositions are not known in the art.

The composition of the present invention may further comprise a pH modifier. The term “pH modifier,” as used herein, means a compound that is capable of changing the pH of a solution. Examples of pH modifiers include, but are not limited to NaOH, KOH, Na2CO3, NaHCO3, K2CO3, KHCO3, NaH2PO4, Na2HPO4, meglumine, Ca(OH)2, Mg(OH)2, pyridoxine, triethanolamine, ammonium hydroxide, cytosine, diethylamine, meglumine, ornithine, glycine, lysine, arginine, aspartic acid, alanine, leucine, diethylethanolamine, guanine, nicotinamide, piperazine, guanidine, olamine, piperidine, triethylamine, tromethamine, benzathine, benzathine, adenine, mixtures thereof and acids, including mineral acids such as hydrochloric acid and organic acids such as acetic acid. Preferred pH modifier for use in present composition includes tromethamine or acetic acid.

In another embodiment, the present invention provides for a process for the preparation of composition comprising
a) preparing a first phase comprising dissolving therapeutically effective amount liraglutide and a block or a graft copolymer or mixtures thereof in water, wherein the first phase forms a gel
b) preparing a second phase comprising dissolving a amphipath or mixture thereof in a water-miscible solvent or mixture thereof, and
c) adding second phase of step b) to first phase of step a) to form the gel composition.

Step a) for the making of composition involves preparing a aqueous phase by dissolving therapeutically effective amount of liraglutide and a block or a graft copolymer or mixtures thereof, in water for injection. This aqueous phase is prepared using the conventional techniques of the pharmaceutical industry which involves dissolving and mixing the ingredients as appropriate to give the desired end product. This aqueous phase forms a gel.

Step b) of the process for making the composition of the invention involves preparation of a second phase comprising dissolving an amphipath or mixture thereof in a water-miscible solvent or mixture thereof. This is prepared by dissolving the amphipath in water-miscible solvents at a temperature of 60 -70° C. under stiffing. The process involves conventional method of mixing by using stirrer. Typically, required amounts of solvents are taken in a tank fitted with a stirrer. Amphipaths are slowly added with stiffing while maintaining the temperature of the mixture at 60 -70° C. Optionally, this second phase may be sterilized by aseptic filtration, preferably by filtering through a 0.2 μ membrane filter.

Step c) of the process involves adding the second phase comprising amphipaths of step b) to a aqueous phase of step a) using stiffing to form a gel.

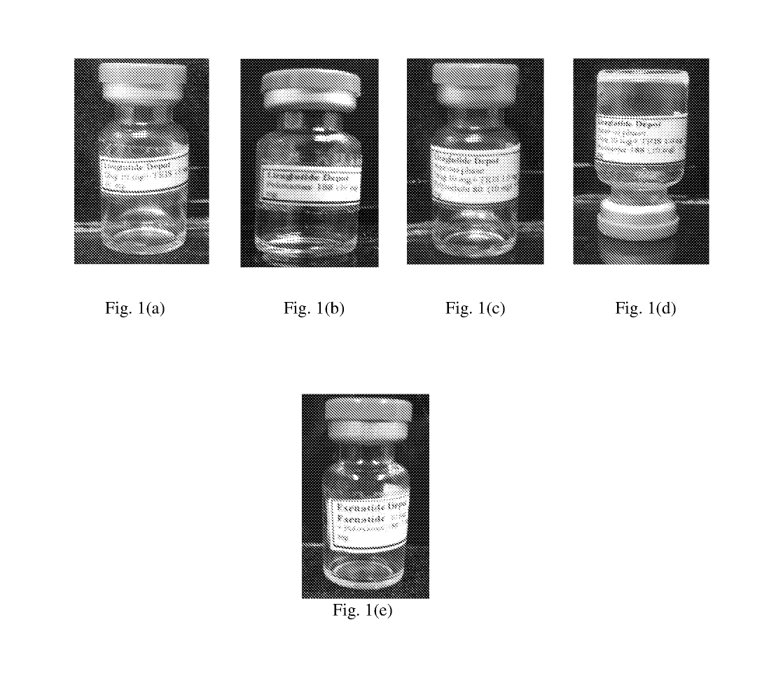

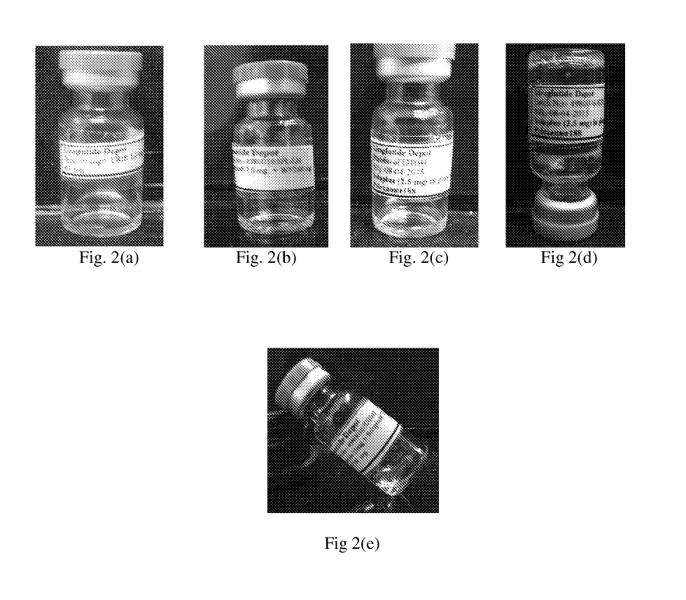

In a preferred embodiment, the composition of the invention can be prepared by a process comprising dissolving a therapeutically effective amount of liraglutide, tromethamine and poloxamer-188/Soluplus® in water for injection. It is observed that the composition of liraglutide as described above forms a gel phase when contacted with water. For eg. Liraglutide when mixed with tromethamine and poloxamer-188 or ^{Soluplus}® forms a gel in water. The gel phase for liraglutide solution is shown in FIGS. 1(d) and 2(d) resp.

In another preferred embodiment, the composition of the invention can be prepared by a process comprising:
a) preparing a first phase comprising dissolving therapeutically effective amount of liraglutide, tromethamine and poloxamer-188/Soluplue in water for injection
b) preparing a second phase comprising mixing glycerol monooleate or a mixture of

Glycerol monoleate, glycerol dioleate, glycerol trioleate and phosphotidylcholine, with ethanol and propylene glycol
c) adding second phase to first phase

The compositions of the present invention are highly advantageous in that the composition provides therapeutically effective levels of liraglutide over 6 days to 14 days. Particular embodiments are administered once weekly. The compositions when administered at therapeutically effective amounts cause significant reduction in blood glucose levels from baseline levels. Further, mean glucose reduction was comparable or better than Victoza®.

Further, the compositions of the invention are also effective in reducing Hb1Ac levels as well as increase in beta cell mass. In addition, the compositions of the invention are also effective in reducing body weight. Thus, the compositions of the present invention are useful for prevention or treatment of type 2 diabetes, hyperglycemia or impaired glucose tolerance as well as for treatment of metabolic diseases like obesity.

The composition of the invention may be administered by injection. In another embodiment, the composition of the invention may be administered by subcutaneous or intramuscular injection.

### EXAMPLES

The compositions of the present invention example are described in detail. However, it is to be noted that the present disclosure is not limited to the illustrative examples but can be realized in various other ways.

### Comparative Example 1

| | | Quantity | |
|---|---|---|---|
| Sr. No. | Ingredients | (mg) | % (w/w) |
| A | Phase I | | |
| 1 | Liraglutide | 5 | 3.91 |
| 2 | Tromethamine | 0.5 | 0.39 |
| 3 | Polysorbate-80 | 2.5 | 1.5 |
| 4 | Water for Injection | 20 | 15.6 |
| | Total Phase I | 28 | |
| B | Phase II | | |
| 1 | Soy Phosphatidyl choline (SPC) | 42.5 | 33.2 |
| 2 | Glycerol Oleates in ratio: - | 42.5 | 33.2 |
| | GMO:GDO:GTO = 44:42:9 | | |
| 3 | Ethanol | 10 | 7.81 |
| 4 | Propylene Glycol | 5 | 3.9 |
| | Total Phase II | 100 | — |
| C | Phase III: Concentrated gel phase | | |
| | (Mixture of Phase I and Phase II): | | |
| 1 | Phase I | 28 | — |
| 2 | Phase II | 100 | — |
| | Total concentrated gel phase | 128 | — |

Manufacturing process of composition:
(A) Phase I: Liraglutide (5 mg), tromethamine (0.5 mg) and polysorbate-80 (2.5 mg) was dissolved in water for injection (20 mg) and kept at 20-25° C.
(B) Phase II: Soy Phosphatidyl choline (SPC) (42.5 mg), Glycerol Oleate (mixture of mono, di and tri oleate and free fatty acid) (Glyceryl monooleate (GMO):Glyceryl diooleate (GDO):Glyceryl trioleate (GTO) = 44:42:9) (42.5 mg), Propylene Glycol (5 mg), Ethanol (10 mg) ware mixed and dissolved at 60-70° C. temperature under stirring for15-20 min.
(C) Phase III: Concentrated gel phase (Mixture of Phase I and Phase II): Phase II was added to phase I using stirring at 60-70° C. temperature to form concentrated gel phase.

### Example 2

Interaction Study of acylated and non-acylated GLP-1 agonist with polyoxyethylene-polyoxypropylene block copolymer-Acylated GLP-1 agonist like liraglutide forms a gel with polyoxyethylene-polyoxypropylene block copolymer in an aqueous vehicle. However, non-acylated GLP-1 agonist like exenatide does not form a gel and remains in solution phase in an aqueous vehicle as seen in FIG. 1(a)-1(e).

### Example 3

Interaction Study of acylated and non-acylated GLP-1 agonist with polyvinyl polyvinyl caprolactam-polyvinyl acetate-polyethylene glycol graft copolymer, Soluplus®-Acylated GLP-1 agonist like liraglutide forms a gel with ^{Soluplus}® in an aqueous vehicle. However, non-acylated GLP-1 agonist like exenatide does not form a gel and remains in solution phase in an aqueous vehicle as seen in FIG. 2(a)-2(e)

### Example 4

| | | Quantity | |
|---|---|---|---|
| Sr. No. | Ingredients | (mg) | % (w/w) |
| A | Phase I | | |
| 1 | Liraglutide | 5 | 3.91 |
| 2 | Tromethamine | 0.5 | 0.39 |
| 3 | Poloxamer 188 (LUTROL F 68) | 2.5 | 1.5 |
| 4 | Water for Injection | 20 | 15.6 |
| | Total Phase I | 28 | |
| B | Phase II | | |
| 1 | Soy Phosphatidylcholine (SPC), | 42.5 | 33.2 |
| | (Phospholipon ® 90 G - Pure | | |
| | phosphatidylcholine stabilized | | |
| | with 0.1% ascorbyl palmitate) (Lipoid) | | |
| 2 | Glycerol Oleates in ratio: - | 42.5 | 33.2 |
| | GMO:GDO:GTO = | | |
| | 44:42:9 (IMWITOR ® 948) | | |
| 3 | Ethanol Absolute 99.9% | 10 | 7.81 |
| 4 | Propylene Glycol USP | 5 | 3.9 |
| | Total Phase II | 100 | |
| C | Phase III: Concentrated gel phase | | |
| | (Mixture of Phase I and Phase II): | | |
| 1 | Phase I | 28 | |
| 2 | Phase II | 100 | |
| | Total concentrated gel phase | 128 | |

Manufacturing process of composition:
(A) Phase I: Liraglutide (5 mg), tromethamine (0.5 mg) and Poloxamer-188 (2.5 mg) was dissolved in water for injection (20 mg) and kept at 20-25° C.
(B) Phase II: Soy Phosphatidylcholine (Phospholipon ® 90 G) (42.5 mg), Glycerol Oleate (mixture of mono, di and tri oleate and free fatty acid) (Glyceryl monooleate (GMO):Glyceryl diooleate (GDO):Glyceryl trioleate (GTO) = 44:42:9) (IMWITOR ® 948) (42.5 mg), Propylene Glycol (5 mg), Ethanol (10 mg) were mixed and dissolved at 60-70° C. temperature under stirring for 15-20 min.
(C) Phase III: Concentrated gel phase (Mixture of Phase I and Phase II): Phase II was added to phase I using stirring at 50-70° C. temperature, to form concentrated gel phase. The lipid gel was yellowish, viscous of semi-solid consistency.

### Example 5

| | | Quantity | |
|---|---|---|---|
| Sr. No. | Ingredients | (mg) | (% w/w) |
| A | Phase I | | |
| 1 | Liraglutide | 5 | 3.91 |
| 2 | Tromethamine | 0.5 | 0.39 |
| 3 | Poloxamer 188 (LUTROL 68) | 2.5 | 1.5 |
| 4 | Water for Injection | 20 | 15.6 |
| | Total Phase | 28 | — |
| B | Phase II | | |
| 1 | Glycerol monooleate | 85 | 66.4 |
| 2 | Ethanol Absolute 99.9% | 10 | 7.8 |
| 3 | Propylene Glycol USP | 5 | 3.9 |
| | Total Phase | 100 | |
| C | Phase III: Concentrated gel phase | | |
| | (Mixture of Phase I and Phase II): | | |
| 1 | Phase I | 28 | |
| 2 | Phase II | 100 | |
| | Total concentrated gel phase | 128 | |

Manufacturing process of composition:
(A) Phase I: Liraglutide (5 mg), tromethamine (0.5 mg) and Poloxamer-188 (2.5 mg) were dissolved in water for injection (20 mg) and kept at 20-25° C.
(B) Phase II: Glycerol monooleate (85 mg), Propylene Glycol (5 mg), Ethanol (10 mg) were mixed and dissolved at 60-70° C. temperature under stirring for 15-20 min.
(C) Phase III: Concentrated gel phase (Mixture of Phase I and Phase II): Phase II was added to phase I using stirring at 50-70° C. temperature to form concentrated gel phase.

### Example 6

Morphology of gel composition of Example 4 and Example 5 when dispersed in WFI was examined using transmission electron microscopic (TEM) technique.

Gel dispersion in WFI

| | | |
|---|---|---|
| Concentrated gel phase | 128 | Mg |
| Water for injection (PH between 6.0 to 7.0) | 872 | Mg |
| Total (final pH obtained 7.0-7.5) | 1000 | Gm |

FEI's Tecnai (TM) Spirit cryo-transmission electron microscope was used for morphology study. The sample of gel dispersion formulation was dropped on standard carbon coated copper grid (mesh) and air dried. TEM images were viewed under transmission electron microscope operating at an accelerated voltage of 120 kV and analyzed.

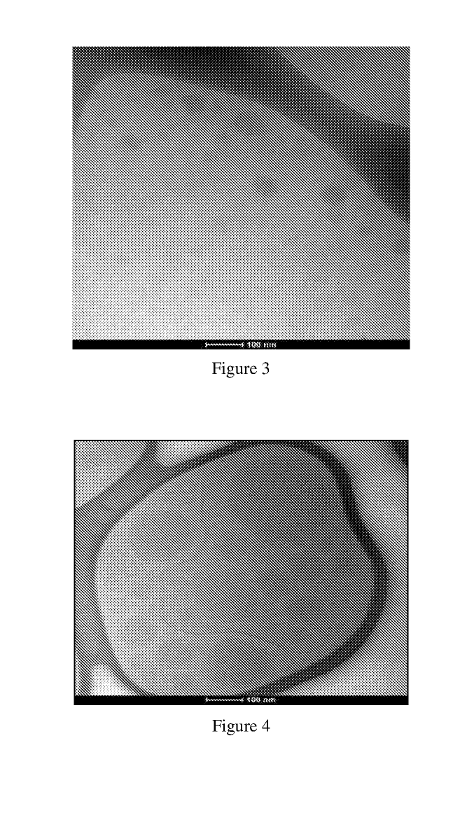

The gel composition of Example 4 and Example 5 shows cubic structure forms when gel is dispersed into the water for injection (See FIGS. 3 and 4).

### Example 7

| | | Quantity |
|---|---|---|
| Sr. No. | Ingredients | (% w/w) |
| | Phase I | |
| 1 | Liraglutide | 6.97 |
| 2 | Tromethamine | 0.7 |
| 3 | Poloxamer | 1.74 |
| 4 | Water for injection | 20.91 |
| | Phase II | |
| 5 | Soy Phosphatidylcholine | 29.62 |
| 6 | Glycerol Oleate (IMWITOR ® 948), Ratio: | 29.62 |
| | GMO:GDO:GTO = 44:42:9 | |
| 7 | Ethanol Absolute 99.9% | 6.97 |
| 8 | Propylene Glycol USP, | 3.48 |
| | Total gel phase (Phase I + Phase II) | 100 |

The gel compositions are prepared according to the same method as Example 4. The gel was yellowish, viscous of semi-solid consistency.

### Example 8

| | | Quantity |
|---|---|---|
| Sr. No. | Ingredients | (% w/w) |
| | Phase I | |
| 1 | Liraglutide | 10 |
| 2 | Tromethamine | 1.25 |
| 3 | Poloxamer | 1.56 |
| 4 | Water for injection | 24.90 |
| | Phase II | |
| 5 | Soy Phosphatidylcholine | 26.48 |
| 6 | Glycerol Oleate (IMWITOR ® 948), Ratio: | 26.48 |
| | GMO:GDO:GTO = 44:42:9 | |
| 7 | Ethanol Absolute 99.9% | 6.23 |
| 8 | Propylene Glycol USP, | 3.10 |
| | Total gel phase (Phase I + Phase II) | 100 |

The gel compositions are prepared according to the same method as Example 4. The gel was yellowish, viscous of semi-solid consistency.

### Example 9

| | | Quantity |
|---|---|---|
| Sr. No. | Ingredients | (% w/w) |
| 1 | Liraglutide | 3.91 |
| 2 | Tromethamine | 0.39 |
| 3 | Poloxamer | 1.95 |
| 4 | Water for Injection | 15.63 |
| 5 | Soy Phosphatidylcholine (Lipoid S 100) | 33.20 |
| 6 | Glycerol Oleates in ratio: - | 33.20 |
| | GMO:GDO:GTO = | |
| | 44:42:9 (IMWITOR ® 948) | |
| 7 | Glycofurol | 7.81 |
| 8 | Propylene Glycol USP | 3.91 |
| | Total gel phase | 100 |

The gel compositions are prepared according to the same method as Example 4. This composition when tested as in Example 10 provided a control on blood glucose levels over a duration of upto 8.5 days.

### Example 10

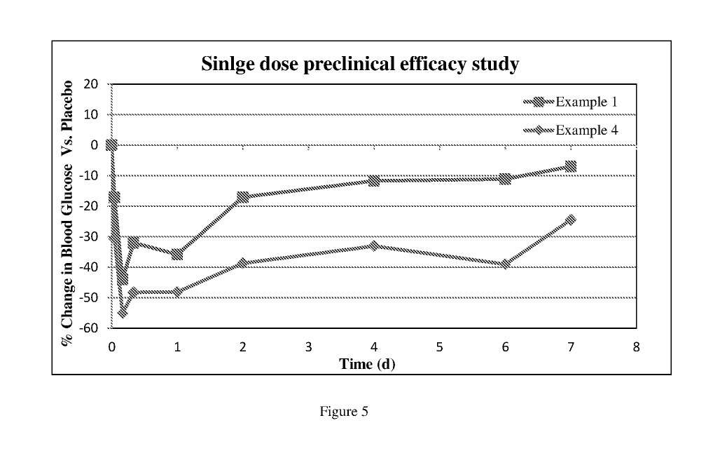

Preclinical efficacy study was performed on the db/db mice model of type 2 diabetes. The animals were acclimatized for 5 days. On day 0, each animal was weighed. The baseline value was determined by collecting approximately 100 μL of blood from retro-orbital plexus and the glucose concentration in the blood was measured with glucose strips Blood Glucose Meter (One Touch™ Ultra198 ; LIFESCAN, Johnson & Johnson). The animals were randomized into treatment groups containing 4 male and 4 female animals each. The dose volume for each animal was calculated based on their respective body weights. A single/multiple dose of composition of Comparative Example 1 and Example 4 were injected subcutaneously in the neck region of the animals. The blood was collected at specific time intervals after post injection. The blood glucose levels were measured as above. The statistical analysis was done on PRISM software. It was observed that Example 4 gave significantly better glucose lowering effects for 7 days compared to Comparative Example 1 as demonstrated in FIG. 5.

### Example 11

Preclinical efficacy study was performed as per the procedure described in Example 10. Compositions of Example 4 and Example 7 were compared with Victoza®. Victoza® was injected daily for 28 days at a human eq. dose of 0.3 mg/kg (1.8 mg is max. approved dose of Victoza®). The blood was collected on 0 d (0, 2, 6, 12 h), 1 d, 2 d, 4 d, 6 d, 7 d, 10 d, 14 d (0, 2, 6, 12 h), 15 d, 17 d, 21 d, 24 d, 28 d (0, 2, 6, 12 h) and 29 d intervals post injection and blood glucose levels was measured. Composition of Example 4 and Example 7 were injected weekly on day 0, 7, 14, and 21 at 10 mg/kg liraglutide (5 times human eq. dose). The blood was collected at 0 d (1 h, 4 h, 8 h, 1 d, 2 d, 4 d, 7 d predose), 7 d (1 h, 4 h, 8 h, 8 d, 9 d, 12 d, 14 d predose), 14 d (1 h, 4 h, 8 h, 15 d, 17, 19, 21 d pre-dose) and 21 d (1 h, 4 h, 8 h, 22 d, 23 d, 25 d, 28 d intervals post injection and blood glucose level was measured. Body weights were measured at various time intervals. HbAlc was measured at initial and at end of the study (28 d) using Kits (Hemoglobin ACl kits, BioSystem, Spain). After last time point, all the animals were sacrificed by CO2 asphyxiation. Pancreas was collected from all the animals and was weighed. Tissue was then placed in a tube containing 10% buffered formalin and transferred sectioning. The tissue sections were then stained using aldehyde fuchsin staining technique to stain β-cell. β-cell count was taken using light microscope and β-cell mass/mg pancreas was calculated (Total no. of β-cells per tissue/mg of pancreas).

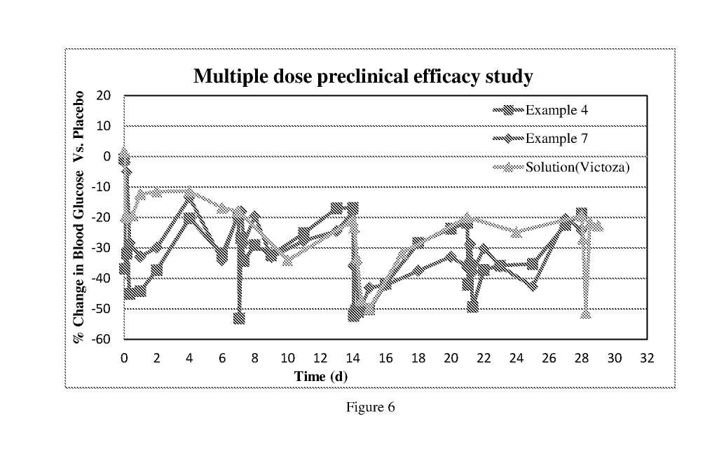

The compositions of Example 4 and Example 7 and solution (Victoza®) showed significant reduction in % blood glucose levels up to 4-weeks. Mean % glucose reduction for Vicotza was 25%, whereas reduction for composition of the invention was about 31% as shown in FIG. 6 and table below.

Mean Glucose reduction, %

| Solution | | |
|---|---|---|
| (Victoza ®) | Example 7 | Example 4 |
| 24.36 | 30.67 | 30.95 |

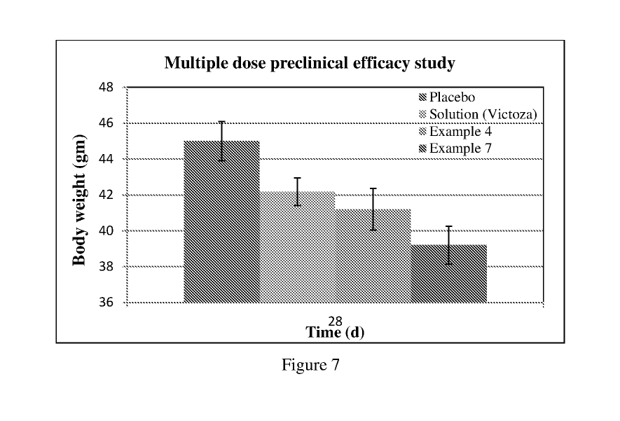

The composition of Example 7, Example 4, and Solution (Victoza®) showed loss in bodyweight of 5.8 gm, 3.8 gm and 2.83 gm respectively after the 4-weeks treatment compared to placebo. The compositions of the invention thus is more effective in reducing body weight compared to the daily solution (Victoza®) as shown in FIG. 7.

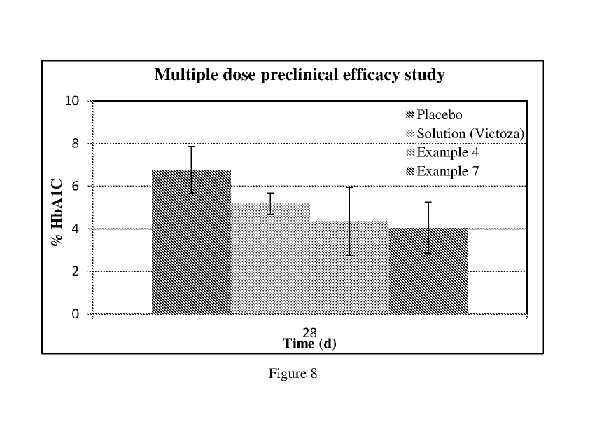

The HbAlC results are shown in FIG. 8, also demonstrates the efficacy of the composition of the invention in reducing HbAlc values. HbAlC reduction was 2.72%, 1.83% and 1.59% respectively for Example 7, Example 4 and Victoza® compared to placebo.

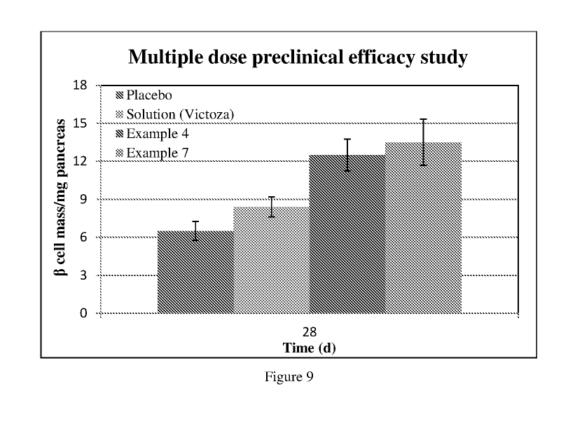

The increase in beta-cell mass was measured initially and at the end of 28 day. Composition of Example 7, Example 4 and Solution (Victoza®) showed increased in total no. of βB-cells of 7, 6 and 1.9 per mg of pancreas respectively after the 4-weeks treatment compared to placebo as shown in FIG. 9.

We claim:

1. A long acting composition comprising:
a) therapeutically effective amount of liraglutide
b) a block or a graft copolymer comprising hydrophilic and hydrophobic moieties or mixtures thereof,
c) atleast one amphipath, and
d) an aqueous vehicle.
wherein the composition is in the form of a gel which is rendered injectable when forced through a needle by means of a plunger.

2. A composition of claim 1, wherein liraglutide is present in a concentration from 3% to 15% by weight of the composition.

3. A composition of claim 2, wherein a block copolymer is selected from polyoxyethylene-polyoxypropylene block copolymer, poly(vinyl alcohol) and poly(ethylene glycol) copolymer, poly(lactic acid) and poly(ethylene glycol) copolymer, Poly(ethyleneoxide)-poly(butadiene)copolymer, Poly(ethyleneoxide)-Poly(ethylene ethylene) copolymer, Dextran-block-poly(ϵ-caprolactone) copolymer.

4. A composition of claim 3, wherein a block copolymer is polyoxyethylene-polyoxypropylene block copolymer

5. A composition of claim 4, wherein the block copolymer is polaxamer 188.

6. A composition of claim 4, wherein polyoxyethylene-polyoxypropylene block copolymer is present at a concentration from 1.5% to 3% by weight of the composition.

7. A composition of claim 2, wherein a graft copolymer is polyvinyl polyvinyl caprolactam-polyvinyl acetate-polyethylene glycol graft copolymer.

8. A composition of claim 2 wherein the amphipath is present at a concentration from 50% to 80% by weight of the composition.

9. A composition of claim 2, wherein the amphipath is selected from glyceryl monooleate, glyceryl dioleate, glyceryl trioleate, polyglyceryl-3-dioleate, phosphotidylcholine and mixtures thereof.

10. A composition of claim 9, wherein the amphipath is a mixture of glyceryl monooleate, glyceryl dioleate, glyceryl trioleate and phosphotidylcholine.

11. A composition of claim 2 wherein the aqueous vehicle is present at a concentration from 20% to 40% by weight of the composition.

12. A composition of claim 11, wherein the aqueous vehicle is a mixture of water and a water miscible solvent

13. A composition of claim 12, wherein water is present at a concentration from 10% to 25% by weight of the composition.
